(19) United States
(12) Patent Application Publication
(10) Pub. No.: US 2017/0276599 A1
(43) Pub. Date: Sep. 28, 2017

(54) COST EFFECTIVE BATTERY-POWERED SPECTROPHOTOMETRIC SYSTEM

(71) Applicant: XiuJun Li, El Paso, TX (US)

(72) Inventor: XiuJun Li, El Paso, TX (US)

(73) Assignee: THE BOARD OF REGENTS OF THE UNIVERSITY OF TEXAS SYSTEM, Austin, TX (US)

(21) Appl. No.: 15/468,186

(22) Filed: Mar. 24, 2017

Related U.S. Application Data
(60) Provisional application No. 62/312,880, filed on Mar. 24, 2016.

(51) Int. Cl. G01N 21/27; B01L 7/00; C12Q 1/68; B01L 3/00

(52) U.S. Cl. CPC G01N 21/272; B01L 3/502715; B01L 7/52; C12Q 1/6844; C12Q 1/689; B01L 2300/0654; C12Q 2600/158

(57) ABSTRACT

Certain embodiments are directed to a low-cost battery-powered spectrophotometric system (BASS) coupled with a microfluidic chip for POC analysis, as well as methods of using the same.

### PRIORITY CLAIM

This application claims priority to U.S. application Ser. No. 62/312,880 filed Mar. 24, 2016, which is incorporated herein by reference in its entirety.

### STATEMENT REGARDING FEDERALLY FUNDED RESEARCH

None.

### BACKGROUND

Point-of-care (POC) analysis is designed to move testing out of well-equipped laboratories into other less hospitable locations. The capacity for on-site or in-field testing from POC analysis is vital for immediate and convenient human health diagnostics, food safety monitoring and environmental analysis (Gubala et al., Analytical chemistry, 2011, 84:487-515). Microfluidic lab-on-a-chip, with a variety of advantages such as low cost and low-reagent consumption associated with portability, miniaturization, integration and automation, offers a versatile miniaturized platform for various applications (Dou et al., Talanta, 2015, 145:43-54; Sanjay et al., Analyst, 2015, 140:7062-81; Dou et al., Analytical chemistry, 2014, 86:7978-86; Zuo et al., Lab Chip, 2013, 13:3921-28; Li et al., Bioanalysis, 2012, 4:1509-25; Li and Zhou, Microfluidic devices for biomedical applications, Elsevier, 2013), providing great potential for POC detection (Chin et al., Lab on a Chip, 2012, 12:2118-34; Yetisen et al., Lab on a Chip, 2013, 13:2210-51; Sun et al., Chemical Society Reviews, 2014, 43:6239-53). However, this great potential is often hindered by conventional detection systems, because most of these systems are devised for cuvette- or glassware-based assays in well-equipped laboratory settings. Therefore, advances in detection systems must be made to fully take the advantages of lab-on-a-chip systems for POC analysis.

Spectrophotometry, quantitative measurement of the reflection, transmission or absorption properties of a compound as a function of light wavelength, is one of the most widely used detection principles in analysis of biological compounds, food analysis, environmental analysis, pharmaceutical analysis, etc. (Ojeda and Rojas, Microchemical Journal, 2013, 106:1-16). However, commercial spectrophotometric systems are generally expensive and bulky, making them only suitable for well-equipped laboratories. Although several portable spectrometers have been reported recently (Ma et al., Journal of hazardous materials, 2012, 219:247-52; Goto et al., Breeding science, 2014, 63:489; Zhang et al., Sensors & Transducers, 2013, 148:47), most of them are cuvette-based and dependent on external AC power supplies. Most cuvette-based systems require large amounts of precious reagents and samples. However, sometimes it is hard to obtain large amounts of biological samples, such as clinical biopsy samples or trace forensic samples from crime scenes. These features make it challenging for the cuvette-based systems to perform POC analysis such as in-field disease diagnosis in low-resource settings where AC electricity is usually not available. Given the advantages of microfluidic lab-on-a-chip, a portable spectrometer coupled with a microfluidic device could significantly reduce the reagent consumption to make it suitable for POC bioanalysis. Jiang and his colleagues integrated an optical detection unit which included commercial optical fibers and a digital fiber optical sensor on a microfluidic chip to measure the real-time absorbance of the turbidity generation from loop-mediated isothermal amplification (LAMP) for quantitative pathogen detection (Fang et al., Analytical Chemistry, 2010, 82:3002-06; Fang et al., Analytical Chemistry, 2010, 83:690-95). However, two thin optical fibers (200 μm diameter core) needed to be inserted in the chip laterally and to be accurately aligned together with the sample chamber, making the whole system complicated. Because the optical fiber cables were fixed in the device, it was not practical to use this detection system for various microfluidic devices, limiting its broad application. Additionally, this detection system still relied on external AC power supplies. These limitations hinder its application for POC analysis in low-resource settings.

### SUMMARY

To address the problems outlined above, the inventor developed a low-cost battery-powered spectrophotometric system (BASS) coupled with a microfluidic chip for POC analysis. In certain aspects the system does not rely on external power supplies. All these features make the spectrophotometric system highly suitable for a variety of POC analyses, such as field detection.

Certain embodiments are directed to a battery-powered spectrophotometric system comprising: (A) a detector, configured to detect light; and (B) a dark box having a bottom, sides, and a cover that when in an operating position form a closed box, wherein (i) the detector is optically coupled to the dark box by one or more optical fibers, (ii) the bottom of the dark box is configured to provide a light source, (iii) the cover is configured to hold a microfluidic device comprising a detection zone; and provide for alignment of the light source, the detection zone, and the optical fiber. In certain aspects the system is powered by a battery. In a further aspect the battery is a 9.0 V battery. In still a further aspect the battery is the battery of a laptop computer or other portable computing device. The system can further comprise a computing device configured to receive data from the detector, transform the data, and provide results based on the data. In other aspects the system can further comprise a microfluidic device incubator configured to develop assay reagents that are applied to or included in the microfluidic device. In certain aspects the assay reagents are nucleic acid amplification reagents. In a further aspect the assay reagents are LAMP reagents, wherein a developed LAMP reaction produces a detectable signal upon the presence of a target nucleic acid. In certain aspects the microfluidic device incubator is a heater. In certain aspect the heater, the dark box, or both the heater and dark box are powered by a battery, e.g., a 9.0 V battery.

Certain embodiments are directed to the use of the system described herein for detecting the presence or absence of a target nucleic acid in a sample.

The phrase “specifically binds” or “specifically immunoreactive” to a target refers to a binding reaction that is determinative of the presence of the molecule, microbe, or other targets in the presence of a heterogeneous population of other biologics. Thus, under designated conditions, a specified molecule binds preferentially to a particular target and does not bind in a significant amount to other biologics present in the sample.

As used herein, the term “test sample” generally refers to a material suspected of containing one or more targets. The test sample may be used directly as obtained from the source or following a pretreatment to modify the character of the sample. The test sample may be derived from any biological source, such as a physiological fluid, including, blood, interstitial fluid, saliva, ocular lens fluid, cerebral spinal fluid, sweat, urine, milk, ascites fluid, mucous, synovial fluid, peritoneal fluid, vaginal fluid, amniotic fluid or the like. The test sample may be pretreated prior to use, such as preparing plasma from blood, diluting viscous fluids, and the like. Methods of treatment may involve filtration, precipitation, dilution, distillation, mixing, concentration, inactivation of interfering components, and the addition of reagents. Besides physiological fluids, other liquid samples may be used such as water, food products, and the like for the performance of environmental or food production assays. In addition, a solid material suspected of containing the target may be used as the test sample. In some instances it may be beneficial to modify a solid test sample to form a liquid medium or to release a target.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Setup of the battery-powered spectrophotometric system for microfluidic devices.

FIGS. 2A-2B. (FIG. 2A) 3D schematic of the microfluidic poly(methyl methacrylate) (PMMA) chip. (FIG. 2B) Photographs of different concentrations of methylene blue in microcentrifuge tubes (left, 1.5-50.0 μM) and in the microfluidic chip (right, 6.2-100.0 μM).

FIGS. 3A-3B. (FIG. 3A) Setup of the commercial spectrophotometric system. (FIG. 3B) Calibration curve of the commercial spectrophotometric system for detection of various concentrations of methylene blue solutions. Inset is a photograph of the commercial external AC electricity-powered EcoVis lamp.

FIGS. 4A-4D. Design of the dark box. (FIG. 4A) Outside view; (FIG. 4B) Inside view of the device holder chamber; (FIG. 4C) Circuit for the LED light assembled in the bottom chamber. (FIG. 4D) Circuit output over a period of time.

FIG. 5. Calibration curve of the battery-powered spectrophotometric system for detection of various concentrations of methylene blue solutions. Inset is a photograph of the dark box.

FIGS. 6A-6B. LAMP detection and subsequent quantitative nucleic acid analysis using the battery-powered spectrophotometric system. (FIG. 6A) Photographs of turbidity-based LAMP detection in microcentrifuge tubes (left) and the microfluidic chip (right). (FIG. 6B) Calibration curve for on-chip LAMP detection and quantitative nucleic acids analysis using the BASS.

### DESCRIPTION

Certain embodiments are directed to a fully battery-powered low-cost spectrophotometric system for quantitative POC analysis on a microfluidic chip. Compared to a commercial spectrophotometric system, the portable spectrophotometric system described herein has at least five significant features: (1) The spectrophotometric system is fully battery-powered without relying on external AC electricity. This feature is significant for POC analysis and in-field detection in resource-poor settings. (2) The spectrophotometric system is cost effective. The dark box can be less than $20. (3) Although no expensive and complicated equipment was used, spectrophotometric system described herein exhibits high detection sensitivity. The on-chip LAMP detection demonstrated that the detection sensitivity is approaching that of Nanodrop, which can cost more than $10,000. (4) The system described herein is compatible with various microfluidic devices, and thus can be used as a universal spectrophotometric detection system for broad applications on microfluidic devices. Additionally, the use of microfluidic devices in the spectrophotometric system significantly reduces the reagent consumption to the microliter level, which is especially important for nucleic acid analysis and clinical testing that usually only limited amounts of samples are available. (5) LAMP detection and subsequent nucleic acid analysis will enable wide applications of the spectrophotometric system due to the widely-used DNA testing techniques. All these features make the system described herein highly suitable for a variety of POC analysis such as human health diagnostics, food safety monitoring and environmental analysis. This is practically significant for developing countries where financial resources are limited.

I. Battery-Powered Spectrophotometric System (BASS)

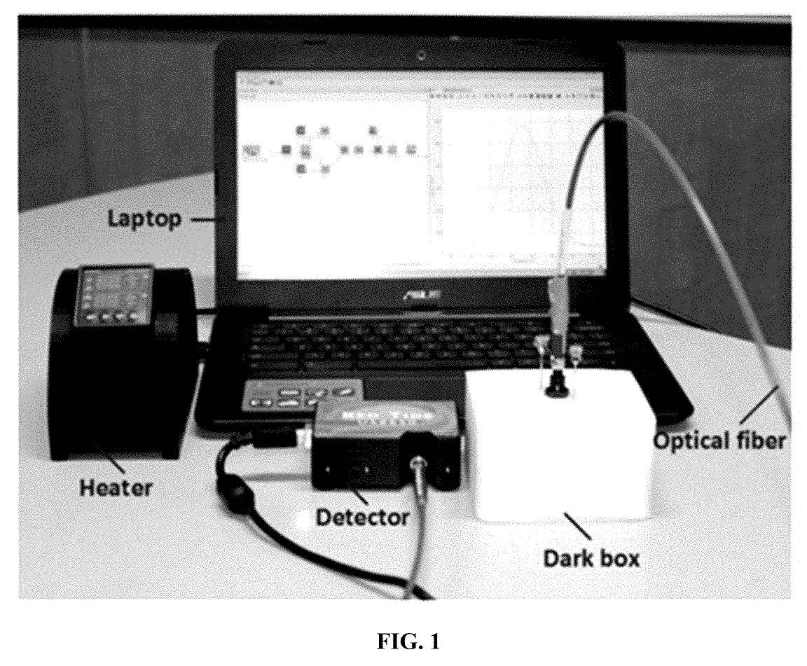

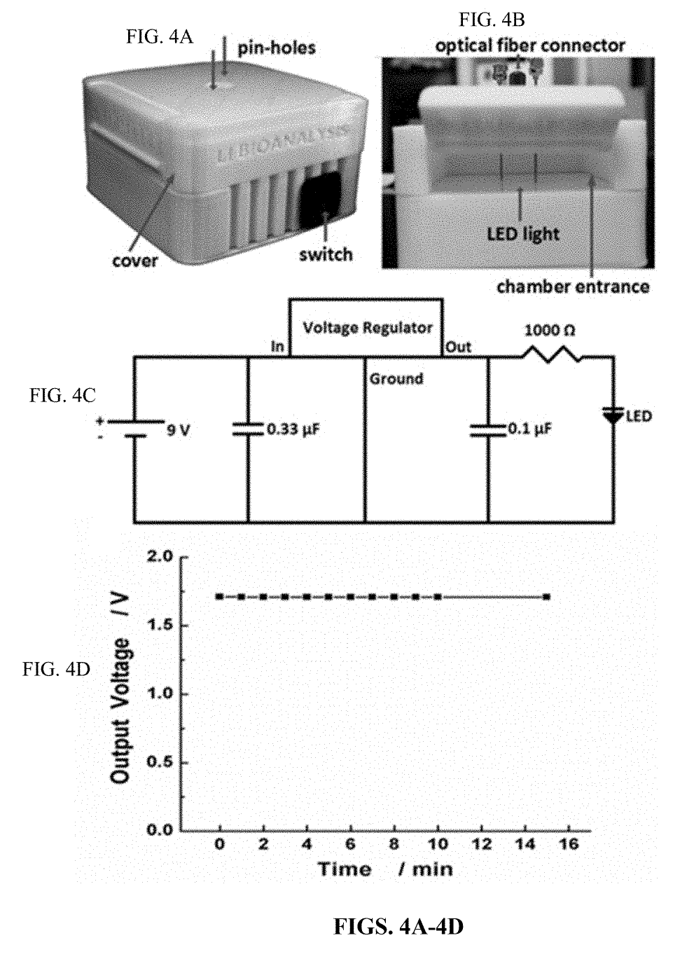

FIG. 1 shows one embodiment of a BASS. In certain aspects the system comprises a dark box configured to expose a device to a light source and collect signal to be evaluated by other components of the system. In certain aspects the dark box can be optically coupled to a detector. One example of a dark box is shown in FIG. 4. In one embodiment the portable dark box (e.g., 10×10×6 cm) has two chambers, with the top chamber serving as the microfluidic device holder and the bottom chamber containing a light, e.g., an LED light, and related circuits as the light source. As can be seen in FIG. 4A and 4B, a cover (i.e., cap) can be 3D-printed to provide access to inside of the dark box. During an assay, the cover is closed to ensure darkness inside, eliminating interference from the environment. On the ceiling of the top chamber, an optical fiber connector can be fixed to connect an optical fiber. In certain aspects two or more pin-holes can be provided to align the optical fiber cable and the detection well of a microfluidic chip with the LED light underneath the top chamber. The pin-hole design is significant for broad applications, because it makes the spectrophotometric system ‘universal’, and readily adapted for various designs of microfluidic devices.

In the bottom chamber, a LED light (e.g., 650 nm red light or other LED light) which can be easily changed to meet the requirement of a number of compounds serves as the light source for the spectrophotometric system, as shown in FIG. 4B. In certain aspects the wavelength of the light can be changed or modulated for various assays. The wavelength of light can include light in the infrared, visible, to ultraviolet wavelengths. The wavelength can be determined based on the adsorption characteristics of the fluorophores, probes, or labels to be detected. In certain aspects an external switch can be provided for turning the light on/off (FIG. 4A). The bottom chamber of the dark box also features a circuit (see FIG. 4C) that provides a steady output voltage of 1.7 V, when, for example, a battery such as a 9.0 V battery is used as the power source. In certain instances it was observed that the output voltage decreased quickly in the absence of a voltage regulator. In order to adjust and have a stable voltage output from the 9.0 V battery, a 5.0 V regulator was applied to the circuit along with two capacitors (0.33 F and 0.1 F) and a 1000 Ω resistor to acquire a constant voltage output of 1.7 V for an LED light. This configuration is a non-limiting configuration and other configurations providing the same result are contemplated. FIG. 4D shows the steady voltage output after the addition of the voltage regulator in the circuit. This stable voltage minimized systematic variations, allowing for accurate measurement. During an assay, light emitted from the battery-powered LED light passes through the sample in the detection zone of the microfluidic chip and gets absorbed. The transmitted light is sent to the detector via an optical fiber cable. The detector can be connected to a laptop other computing device through a USB cable or other such connector, without any external supplies.

In certain embodiments certain components can be produced using 3D printing. The spectrophotometric system is portable and fully battery-powered to enable on-site or in-field use where or when AC electricity is available.

In certain embodiments a battery-powered spectrophotometric system can include one or more of (i) a light source, (ii) a microfluidic device, (iii) a device holder or dark box, (iv) optical fiber cables, (v) a detector, (vi) a laptop computer, and (vii) one or more power source.

The system described herein can be configured to be compatible with a variety of microfluidic devices. One or more device holder can be included that is compatible with one or more microfluidic device. The device holder can secure the microfluidic device and provide for proper alignment for processing and detection purposes. In certain embodiments of a microfluidic device can be configured to for single- or multiplexed pathogen detection. One such device can have three or more layers. The top layer can be a polymer layer used for reagent delivery, three microchannels (e.g., length 10 mm, width 100 μm, depth 100 μm) are formed in the top layer. Also formed in the top layer is an inlet reservoir (e.g., diameter 1.0 mm, depth 1.5 mm). The middle layer can be a polymer layer having two or more detection zones (e.g., diameter 1.0 mm, depth 1.5 mm, volume ˜4 μL each), outlet reservoirs (e.g., diameter 1.0 mm, depth 1.5 mm) and microchannels (e.g., length 9.5 mm, width 100 μm, depth 100 μm). In certain aspects the detection zone can be a loop-mediated isothermal amplification (LAMP) zone(s) that can be used for LAMP reaction and detection. The bottom layer can be a support layer (e.g., a glass slide (length 75 mm, width 25 μm, depth 1.0 μm). Different detection zones can be used for negative control (NC), positive control (PC), and pathogen detection.

The detection portion of the device can comprise specific primers and/or specific probes for target pathogens or positive control DNA can be pre-loaded or supplied during the processing of a sample in the detection zone. In certain aspects a detection zone can be loaded with 1, 2, 3, 4, or more primer pairs. In certain aspects amplification and detection are performed in the detection zone. In a further aspect, an amplification reaction is transferred to a separate detection zone. A device can be configured to transport a reaction mixture and/or sample from an inlet to fill the detection zone(s). In certain aspects a filter is included in the device and positioned such that a sample being applied to the device is filtered prior to being transported to a detection zone. After filling, the inlet and outlets can be sealed, e.g., with epoxy. Amplification can then performed at an appropriate temperature an appropriate amount of time. Microfluidic devices and systems can be configured to perform a number of different analytical and/or synthetic operations within the confines of very small channels and chambers that are disposed within small scale integrated microfluidic devices. Multiplexing the basic system can substantially increase throughput, so that the operations of the system are carried out in highly parallelized system.

Microfluidic devices and systems are well suited for parallelization or multiplexing because large numbers of parallel analytical fluidic elements can be combined within a single integrated device that occupies a relatively small area. A multiplexing device will comprise a plurality of channels and microwells that are configured to analyze a number of different analytes, such as pathogens.

In certain aspects a microfluidic device can comprise a nucleic acid amplification chamber or detection zone(s), microchannels, and ports. In certain aspects the device can have 1, 2, 3, 4, 5, 6, or more microchannels. The microchannels can be in fluid communication with 1, 2, 3, 4, 5, 6 or more detection zones. Each detection zone can have one or more detectable probes.

In certain aspects the detection zone can be sealed, for example with a tape layer, a cap, or mineral oil to prevent liquid evaporation. DNA in a sample(s) can be isothermally amplified by LAMP or a similar process (Ahmad et al. (2011) Biomed Microdevices, 13(5): 929-37). In certain aspects, a portable heating unit can be included in the system. In one aspect the heating unit can include a proportional-integral-derivative (PID) temperature controller (Auber Inst, Ga.), a thermocouple (Auber Inst.), and a heating film (Omega, CT). During processing a target analyte (e.g., a target nucleic acid) can be labeled with fluorophores or associated with a labeled probe for fluorescence detection.

In certain embodiments the microfluidic device is configured for nucleic acid amplification using LAMP or other isothermal nucleic acid amplification methods. In certain aspects a LAMP method can use Bacillus stearothermophilus DNA polymerase, a thermally-stable enzyme with high displacement ability over the template-primer complex (Saleh et al. (2008) Dis Aquat Organ, 81(2): 143-51; Notomi et al. (2000) Nucleic Acids Res, 28(12): E63). The LAMP amplification technique allows nucleic acid amplification to be carried out under thermally constant conditions, eliminating the use of expensive and cumbersome thermal cycler equipment in low-resource settings.

Certain embodiments incorporate a miniaturized portable fluorescence detection system using a light emitting diode (LED), such as violet LED (Tsai et al. (2003) Electrophoresis, 24(17): 3083-88), a UV LED, or a laser pointer. The wavelength of 532 nm from a green laser pointer is a good fit with the excitation wavelength of one of the common probes-Cy3, but other combinations of light source and fluorophore can be used.

Certain embodiments incorporate a visual fluorescent or a colorimetric detection method. Mori et al (2001) observed that during the LAMP amplification process, a magnesium pyrophosphate precipitate was formed as a turbid by-product of the nucleic acid amplification process (Mori et al. (2001) Biochem Biophys Res Commun, 289(1): 150-54). This precipitate forms only when the targeted DNA is present in the LAMP amplification process, such that the presence of the pyrophosphate can serve as an indicator of the presence of a target. In certain aspects an intercalcating dye can be used to detect product amplification (Ji et al. (2010) Poult Sci, 89(3): 477-83).

In certain embodiments a filtration layer can be included to remove red blood cells in order to avoid detection inference in subsequent steps.

Certain configurations of the system can include configurations for receiving microfluidic devices having multiple detection zones. In certain aspects different detection zones will have different primers. When an appropriate target is present a detectable signal is generated and detected and processed by the system. In certain aspect the system will comprise a computer or controller to receive detection data, process the detection date, generate a result, and present the result to receiver. The receiver can be a human or other electronic device configured to manage such information.

In certain embodiments a microfluidic device is configured for meningitis diagnosis in a laboratory or home setting. In other embodiments the system is configured to provide a POC device for field diagnosis. Furthermore, the system and related methods can be used to detect various plant, animal, food-borne, and other infectious diseases (e.g., Bacillus pertussis, HIV, etc.) in resource-limited settings.

Probes can be coupled to a variety of reporter moieties. Reporter moieties include fluorescent reporter moieties that can be used to detect probe binding to or amplification of a target. Fluorophores can be fluorescein isothiocyanate (FITC), allophycocyanin (APC), R-phycoerythrin (PE), peridinin chlorophyll protein (PerCP), Texas Red, Cy2, Cy3, Cy3.5, Cy5, Cy5.5, Cy7; or fluorescence resonance energy tandem fluorophores such as PerCPCy5.5, PE-Cy5, PE-Cy5.5, PE-Cy7, PE-Texas Red, and APC-Cy7. Other fluorophores include, Alexa Fluor® 350, Alexa Fluor® 488, Alexa 25 Fluor® 532, Alexa Fluor® 546, Alexa Fluor® 568, Alexa Fluor® 594, Alexa Fluor® 647; BODIPY dyes, such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665; Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, and tetramethylrhodamine, all of which are also useful for fluorescently labeling nucleic acids or other probes or targets.

In certain aspects the fluorescence of a probe can be quenched. Quenching refers to any process that decreases the fluorescence intensity of a given substance. A variety of processes can result in quenching, such as excited state reactions, energy transfer, complex-formation, and collisional quenching. The chloride ion is a well-known quencher for quinine fluorescence. Typically quenching poses a problem for non-instant spectroscopic methods, such as laser-induced fluorescence, but can also be used in producing biosensors. In certain aspects the fluorescence of a labeled probe that is not bound to its target is quenched, wherein upon binding to its target the fluorescence is recovered and can be detected. The labeled probe is complexed with a quenching moiety in the detection zone. Once the probe binds its target the fluorescence is recovered. Target binding results in increased fluorescence.

In certain embodiments, the invention concerns portable, rapid and accurate POC systems for detecting microbes, including without limitation, parasites and their eggs, Noroviruses (Norwalk-like viruses), Campylobacter species, Giardia lamblia, Salmonella, Shigella, Cryptosporidium parvum, Clostridium species, Toxoplasma gondii, Staphylococcus aureus, Shiga toxin-producing Escherichia coli (STEC), Yersinia enterocolitica, Bacillus cereus, Bacillus anthracis, Cyclospora cayetanensis, Listeria monocytogenes, Vibrio parahemolyticus and V. vulnificus. The term “microorganism” or “microbe” as used in this disclosure includes a virus, bacterium, fungi, parasite, or parasite's egg. In certain aspects a pathogenic or potentially pathogenic microbe can be detected. A pathogenic microbe can be a virus, a bacterium, and/or a fungus. In certain aspects the system can be configured to detect a variety of microbes including viruses, bacteria, and fungi simultaneously. In certain aspects, a microbe includes a virus. The virus can be from the Adenoviridae, Coronaviridae, Filoviridae, Flaviviridae, Hepadnaviridae, Herpesviridae, Orthomyxoviridae, Paramyxovirinae, Pneumovirinae, Picornaviridae, Poxyiridae, Retroviridae, or Togaviridae family of viruses; and/or Parainfluenza, Influenza, H5N1, Marburg, Ebola, Severe acute respiratory syndrome coronavirus, Yellow fever virus, Human respiratory syncytial virus, Hantavirus, or Vaccinia virus.

In yet a further aspect, the pathogenic or potentially pathogenic microbe can be a bacteria. A bacterium can be an intracellular, a gram positive, or a gram negative bacteria. In a further aspect, bacteria include, but is not limited to a Neisseria meningitidis (N. meningitidis), Streptococcus pneumoniae (S. pneumoniae), Haemophilus influenzae type B (Hib), B. pertussis, B. parapertussis, B. holmesii, Escherichia, a Staphylococcus, a Bacillus, a Francisella, or a Yersinia bacteria. In still a further aspect, the bacteria is Bacillus anthracis, Yersinia pestis, Francisella tularensis, Pseudomonas aerugenosa, or Staphylococcus aureas. In still a further aspect, a bacteria is a drug resistant bacteria, such as a multiple drug resistant Staphylococcus aureas (MRSA). Representative medically relevant Gram-negative bacilli include Hemophilus influenzae, Klebsiella pneumoniae, Legionella pneumophila, Pseudomonas aeruginosa, Escherichia coli, Proteus mirabilis, Enterobacter cloacae, Serratia marcescens, Helicobacter pylori, Salmonella enteritidis, and Salmonella typhi. Representative gram positive bacteria include, but are not limited to Bacillus, Listeria, Staphylococcus, Streptococcus, Enterococcus, Actinobacteria and Clostridium Mycoplasma that lack cell walls and cannot be Gram stained, including those bacteria that are derived from such forms.

In still another aspect, the pathogenic or potentially pathogenic microbe is a fungus, such as members of the family Aspergillus, Candida, Crytpococus, Histoplasma, Coccidioides, Blastomyces, Pneumocystis, or Zygomyces. In still further embodiments a fungus includes, but is not limited to Aspergillus fumigatus, Candida albicans, Cryptococcus neoformans, Histoplasma capsulatum, Coccidioides immitis, or Pneumocystis carinii. The family zygomycetes includes Basidiobolales (Basidiobolaceae), Dimargaritales (Dimargaritaceae), Endogonales (Endogonaceae), Entomophthorales (Ancylistaceae, Completoriaceae, Entomophthoraceae, Meristacraceae, Neozygitaceae), Kickxellales (Kickxellaceae), Mortierellales (Mortierellaceae), Mucorales, and Zoopagales.

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Batter-Powered Spectrophotometric System

The BASS spectrophotometric system not only required less sample, but also exhibited higher detection sensitivity than the commercial spectrophotometric system. By using methylene blue as a model analyte, the inventor first compared the performance of the BASS with a commercial spectrophotometric system, and further applied the BASS for loop-mediated isothermal amplification (LAMP) detection and subsequent quantitative nucleic acid analysis which exhibited a comparable limit of detection to that of Nanodrop. Compared to the commercial spectrophotometric system, spectrophotometric system described is lower-cost, consumes less reagents, and has a higher detection sensitivity.

Results

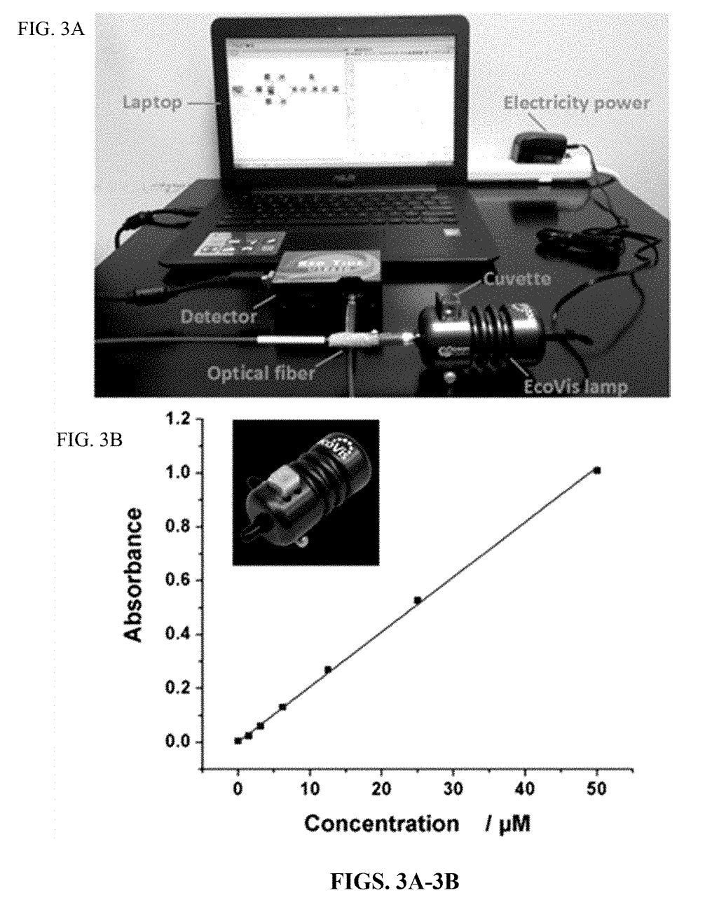

Methylene Blue Measurement Using a Commercial Spectrophotometric System. A commercial spectrophotometric system was also used to compare the performance with the BASS. The setup of the commercial spectrophotometric system is shown in FIG. 3A. The commercial spectrophotometric system uses a commercial EcoVis lamp as the light source (˜$600) which requires external AC power supplies. The EcoVis lamp has a holder (FIG. 3A), but it is only compatible for cuvettes, and not suitable for microfluidic devices. In addition, a relatively large amount of samples (at least 0.8 mL) is required to perform an assay for cuvette-based systems. These features hinder its applications for POC analysis.

Methylene blue, a heterocyclic aromatic chemical compound, has been widely used in biology, chemistry and health science as a staining agent. Methylene blue is also used to treat methemoglobinemia as a pharmaceutical drug (Zhang et al., The Annals of thoracic surgery, 2015, 99:238-42). Methylene blue was used as a model analyte to demonstrate the colorimetric bioassay, and to compare the performance of system described herein with the commercial system. The inventor first conducted the detection of different concentrations of methylene blue using the commercial spectrophotometric system.

FIG. 3B shows the calibration curve plotted by using absorbance versus various concentrations of methylene blue ranging from to 0 to 50.0 μM. With the increase of the methylene blue concentration, stronger absorbance was observed. A linear calibration curve was established between the absorbance and methylene blue concentration, with the square of the correlation coefficient (R²) of 0.9985. The limit of detection (LOD) of methylene blue was calculated to be 0.59 μM on the basis of the 3-fold standard deviations of the negative control signal.

Battery-Powered Spectrophotometric System. FIG. 1 shows the setup of one embodiment of BASS. Since EcoVis requires external power supplies and is not suitable for microfluidic devices, a dark box was developed, a major difference between these two systems, to address these issues for broader POC applications, as shown in FIG. 4.

The portable dark box (e.g., having dimensions of 10×10×6 cm) has two chambers, with the top chamber serving as the microfluidic device holder and the bottom chamber containing a LED light and related circuits as the light source. As can be seen in FIGS. 4A and 4B, a cover (i.e., cap) was 3D-printed to provide access to inside of the dark box. During an assay, the cover was closed to ensure darkness inside, eliminating interference from the environment (No cover is used for the commercial EcoVis lamp). On the ceiling of the top chamber, an optical fiber connector was fixed to connect the optical fiber, and two pin-holes were designed to align the optical fiber cable and the detection well of the microfluidic chip with the LED light underneath the top chamber. The pin-hole design is significant for broad applications, because it makes the spectrophotometric system become ‘universal’, and readily adapted for various designs of microfluidic devices.

In the bottom chamber, a LED light (e.g., 650 nm red light) which can be easily changed to meet the requirement of a number of compounds serves as the light source for the spectrophotometric system, as shown in FIG. 4B. An easy-to-use switch is designed for turning the LED light on/off (FIG. 4A). The bottom chamber of the dark box also features a circuit (see FIG. 4C) that provides a steady output voltage of 1.7 V, when a 9.0 V battery is used as the power source. It was observed that the output voltage decreased quickly in the absence of a voltage regulator. In order to adjust and have a stable voltage output from the 9 V battery, a 5 V voltage regulator was applied to the circuit along with two capacitors (e.g., 0.33 g and 0.1 g) and a resistor (e.g, 1000 Ω) to acquire a constant voltage output of 1.7 V for the LED light. FIG. 4D shows the steady voltage output after the addition of the voltage regulator in the circuit. This stable voltage minimized systematic variations, allowing for accurate measurement. During an assay, light emitted from the battery-powered LED light passes through the sample in the detection zone of the microfluidic chip and gets absorbed. The transmitted light is sent to the detector via the optical fiber cable, while the detector is connected to a laptop computer, e.g., through a USB cable, without any external supplies.

Different components were assembled through 3D printing, with a user-friendly interface. The total material cost of the dark box was estimated less than $20, which was much lower compared to the commercial external AC electricity-powered and cuvette-based EcoVis lamp (˜$600). Most importantly, the spectrophotometric system is portable and fully battery-powered, which is a desirable trait in the evolving market of spectrophotometry for on-site or in-field testing where AC electricity is commonly not available.

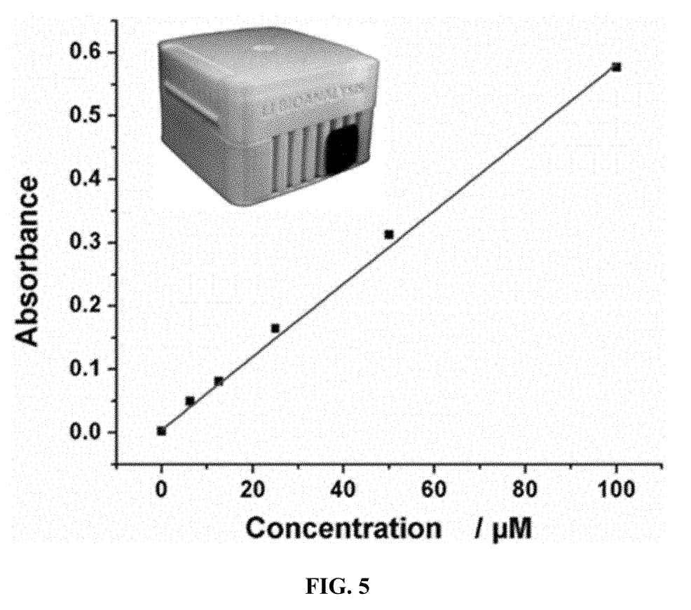

Performance of the battery-powered spectrophotometric system. The inventors then measured various concentrations of methylene blue using the BASS to evaluate its performance. As shown in FIG. 5, a similar linear calibration curve was observed between the absorbance and the concentration of methylene blue in the experimental range (0 to 100.0 μM), with the square of the correlation coefficient (R²) of 0.9987. The LOD was calculated to be as low as 0.10 μM on the basis of the 3-fold standard deviations of the negative control signal. This indicates that the BASS is more sensitive than the commercial spectrophotometric system (LOD 0.59 μM). Given that the optical path of the microfluidic chip is only 1.5 mm while the optical path of the cuvette is 1.0 cm, detection sensitivity could be improved by at least one order of magnitude (˜30 fold herein), if using the same optical path. Along with the impressive performance from a portable spectrophotometric system as described herein, the spectrophotometric system can perform POC analysis on a microfluidic chip with only a tiny amount of sample needed (˜10 μL/assay). Compared to the commercial cuvette-based spectrophotometric system which requires at least 0.8 mL reagent for one assay, microfluidic chip-based spectrophotometric system significantly reduces the reagent consumption.

LAMP Detection and Subsequent Quantitative DNA Analysis using the Battery-Powered Spectrophotometric System. The inventor further exploited the potential application of the BASS by performing DNA-based high-sensitivity pathogen detection through LAMP. LAMP is a simple, rapid, specific and cost-effective nucleic acid amplification method that allows the target DNA sequence amplified at a constant temperature of 60-65° C. (Tomita et al., Nature protocols, 2008, 3:877-82). Avoiding the use of expensive equipment (e.g., thermal cyclers) for stringent thermal cycling, LAMP is more compatible with microfluidic chips without complicated and costly microfabrication of heating elements and temperature sensors, and has great potential for POC disease diagnosis (Dou et al., Analytical chemistry, 2014, 86:7978-86; Fang et al., Analytical Chemistry, 2010, 82:3002-06; Fang et al., Lab on a Chip, 2012, 12:1495-99; Safavieh et al., Analyst, 2014, 139:482-87; Safavieh et al., Biosensors and Bioelectronics, 2012, 31:523-28; Wang et al., Lab on a Chip, 2011, 11:1521-31).

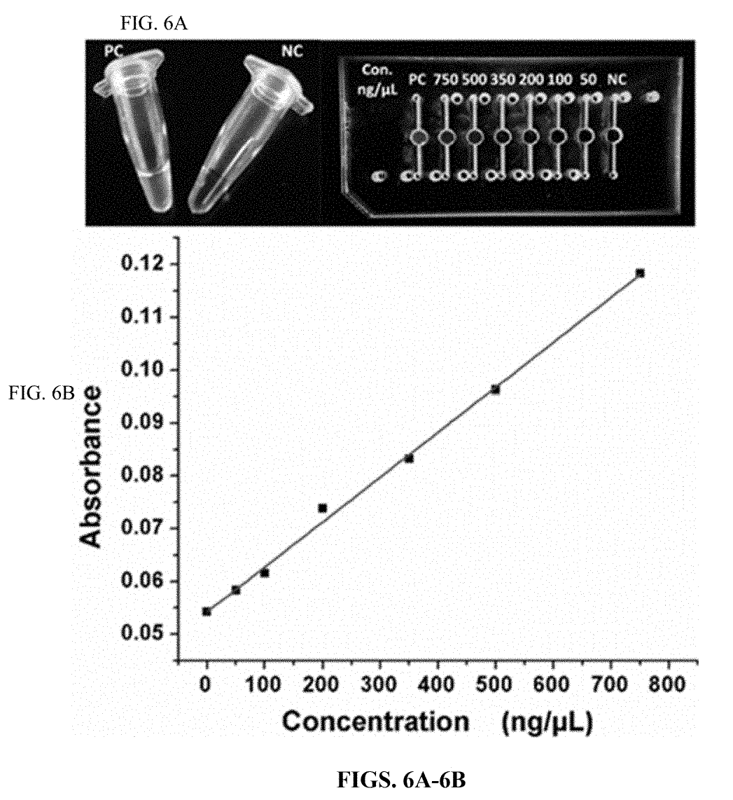

On-chip LAMP was performed for detection of a main meningitis causing bacteria, Neisseria meningitidis, which can be fatal in as little as 24 hours after symptoms are noticed and has a high incidence rate in high-poverty areas (Castillo, WHO Manual, 2nd Edition, 2011). Therefore, a simple, low-cost, highly-sensitive POC analysis is in great need for immediate diagnosis of meningitis. The on-chip LAMP was heated at 63° C. for 45 min by using a portable-battery-powered heater with an inexpensive proportional-integral-derivative (PID)-based temperature controller devised by the inventor's laboratory. During the LAMP process, a byproduct of magnesium pyrophosphate precipitate is formed when the targeted DNA is present. Therefore, the presence of the magnesium pyrophosphate precipitate can serve as an indicator of the presence of the target pathogen's DNA by turbidity tests. However, it is challenging to use turbidity testing for high-sensitivity visual detection. FIG. 6A shows the turbidity visual detection both in microcentrifuge tubes and the microfluidic chip. It showed that the turbidity difference between the positive control (PC) and negative control (NC) could be seen in the tubes, but could barely be seen in the microfluidic chip even at the highest concentration without any dilution (the detection well with PC) which was mainly due to shorter optical path length from the microfluidic device. However, the nucleic acid samples are usually limited, a challenge for cuvette-based spectrophotometric system due to its large amount of reagent requirements.

To address these issues, the inventor performed sensitive LAMP detection and subsequent quantitative nucleic acid quantification on a chip using an embodiment of the portable spectrophotometric system based on turbidity testing at 650 nm. The LAMP products were tested on the microfluidic device and their corresponding absorbance signals were recorded. Excitingly, a clear difference between the LAMP amplicon and the negative control on the microfluidic device was observed (FIG. 5), even though the optical path length from the microfluidic device was short and only 10 μL of samples were needed. The inventor further made a series of dilutions from the LAMP amplicons to study the relationship between the DNA concentration and the absorbance at 650 nm. It was interesting to find that, with the increase of nucleic acid concentration, a stronger absorbance signal was observed. FIG. 6B shows a linear calibration curve plotted by using the corresponding absorbance signals versus various concentrations of nucleic acids from the LAMP products ranging from 0 to 750.0 ng/μL, with the square of the correlation coefficient (R²) of 0.9968. Compared to the absorbance signal of the negative control, even 50.0 ng/μL of nucleic acids showed well distinguishable absorbance signal. The LOD of nucleic acids was calculated to be as low as 15.3 ng/μL on the basis of the 3-fold standards deviations of the negative control signal, which was ˜50 folds lower than the nucleic acid concentration of the undiluted LAMP products. This also indicates the detection sensitivity of our spectrophotometric system is approaching to that of Nanodrop which has a LOD of ˜2.5 ng/μL (Gallagher and Desjardins, Current Protocols in Human Genetics, 2007, A. 3D. 1-A. 3D. 21), whereas the Nanodrop may cost more than $10,000. Therefore, the BASS is capable of achieving high-sensitivity LAMP detection and subsequent quantitative nucleic acid analysis under low-resource settings (e.g. without AC electricity).

Chemicals and Materials

Methylene blue was purchased from Sigma (St. Louis, Mo.). The LAMP reaction mixture contained 20 mM Tris-HCl (pH 8.8), 10 mM KCl, 8 mM MgSO₄, 10 mM (NH₄)₂SO₄, 0.1% Tween 20, 0.8 M Betaine, 0.5 mM MnCl₂, 1.4 mM dNTPs, 8U Bst Polymerase, 1.6 μM each of the inner primer (FIP/BIP), 0.2 μM each of the outer primer (F3/B3), 0.4 μM each of the loop primer (LF/LB). The LAMP primers were purchased from Integrated DNA Technologies (Coralville, Iowa). The Neisseria meningitidis (ATCC 13098) was obtained from American Type Culture Collection (ATCC, Rockville, Md.). LAMP DNA amplification kits were purchased from Eiken Co. Ltd., Japan. DNA isolation kits and LAMP products purification kits were purchased from Qiagen (Valencia, Calif.). LAMP products were collected and purified for concentration measurement of the nucleic acids by using Nanodrop (Nanodrop 1000, Thermo Scientific, Ma.). Unless stated otherwise, all solutions were prepared with ultrapure Milli-Q water (18.2 MΩ cm) from a Millipore Milli-Q system (Bedford, Mass.).

The chip substrate PMMA was purchased from McMaster-Carr (Los Angeles, Calif.). The absorbance detector of Red Tide USB 650, EcoVis lamp and optical fiber cables were purchased from Ocean Optics (Dunedin, Fla.). The red LED light was purchased from Weller (Apex, N.C.). The dark box (10×10×6 cm) and the heater control holder were 3D printed using a 3D printer purchased from MakerBot Industries (Brooklyn, N.Y.).

Experimental Design and Setup

The setup of the battery-powered spectrophotometric system is shown in FIG. 1. The system can include or consist of a LED light source, a microfluidic device, a device holder, optical fiber cables, a detector Red Tide USB 650 and a laptop computer. The light source and related circuits were assembled in the bottom chamber of the 3D printed dark box, while the top chamber of the dark box was designed to hold a microfluidic device. After the light emitted from the LED light passes through a sample in the microfluidic chip, the transmitted light will be sent to the detector via optical cables. Absorbance data will be sent to the computer through a USB cable, and recorded by the software Ocean View installed in a laptop. All the components including the heater controller are fully battery-powered, and do not require external AC power supplies.

A similar commercial spectrophotometric system (Ocean Optics) was used to compare the performance of the spectrophotometric system described herein, using the EcoVis lamp as the light source. The EcoVis lamp's light was attenuated by using a 16 natural density filter to make it suitable for the detector.

Chip Design and Fabrication

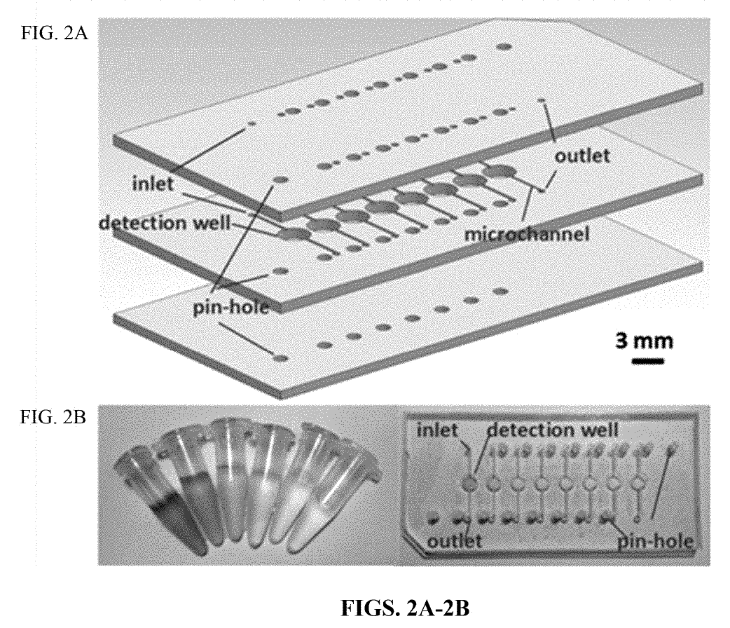

The layout of a simple microfluidic PMMA chip used in the BASS is shown in FIG. 2A. The microfluidic chip comprises three layers. All three layers have corresponding pin-holes to fix the microfluidic chip and allow aligning the detection zone of the chip and optical fiber cables with the LED light underneath (e.g., 650 nm red light). In addition, the top two layers contain inlets and outlets and the middle layer contains microchannels for reagent introduction. All the features including the pin-holes, inlets, outlets, detection wells (diameter 3 mm) and microchannels were directly laser ablated by a laser cutter (Epilog Zing 16, Golden, Colo.) within a few minutes, and then different layers were bonded together after heating in an oven at 120° C. for 30 min. FIG. 2B shows a photograph of the microfluidic chip and 2.5 mL microcentrifuge tubes filled with different concentrations of methylene blue solutions which were used to evaluate the performance between the commercial cuvette-based spectrophotometric system and the BASS.

LAMP Detection Procedures

In order to explore the relationship between the DNA concentrations from the LAMP amplicon with the signal from the BASS, after the LAMP reaction, the amplicon was collected and a series of dilutions made. The nucleic acid concentrations of the standard LAMP products were measured by using Nanodrop. Meanwhile, corresponding absorbance signals from each dilution were measured using the BASS.

1. An inexpensive battery-powered spectrophotometric system comprising:
(a) a detector, configured to detect light; and
(b) a dark box having a bottom, sides, and a cover or top chamber that when in an operating position form a closed box, wherein (i) the detector is optically coupled to the dark box by one or more optical fibers, (ii) the bottom of the dark box is configured to provide a light source, (iii) the cover or a top chamber is configured to hold a microfluidic or millifluidic device and provide for alignment of the light source, microfluidic device, and optical fiber.

2. The system of claim 1, further comprising a removable microfluidic device.

3. The system of claim 2, wherein the microfluidic device further comprises at least one microwell containing a detectable moiety.

4. The system of claim 3, wherein the microwell contains an amplification primer.

5. The system of claim 1, further comprising a computing device configured to receive data from the detector, transform the data, and provide results based on the data.

6. The system of claim 1, further comprising a microfluidic /millifluidic device holder configured to contain assay reagents.

7. The system of claim 1, wherein the light source is a LED light or multiple LED lights with different wavelengths.

8. The system of claim 7, wherein the light source is powered by a battery and related circuits to provide stable output.

9. The system of claim 8, wherein the battery is a 9 volt battery or a battery for a laptop or mobile device.

10. The system of claim 1, wherein the detector is a photomultiplier tube, photodiode or other transducers with or without functions of resolving different light wavelengths.

11. The system of claim 10, wherein the detector is operably coupled to a laptop or other portable computing device.

12. The system of claim 6, wherein the assays are loop-mediated DNA/RNA isothermal amplification (LAMP) assays or other isothermal methods, wherein a developed LAMP reaction produces a detectable signal upon the presence of a target nucleic acid.

13. The system of claim 6, wherein the microfluidic/millifulidic device has pin-hole or other structures to align with the light source and the detector.

14. A method of detecting an analyte comprising introducing a sample into a microfluidic device, incubating the device to produce a detectable product, and interrogating the device using the system of claim 1.

15. The method of claim 14, wherein the analyte is a nucleic acid.

16. The method of claim 15, wherein the nucleic acid is amplified.

17. The method of claim 16, wherein the nucleic acid is amplified using isothermal amplification.

18. The method of claim 14, wherein the sample is a biological fluid.

19. The method of claim 14, wherein the sample is an environmental sample.
